(19) United States
(12) Patent Application Publication
(10) Pub. No.: US 2025/0160884 A1
(43) Pub. Date: May 22, 2025

(54) MEDICAL DEVICE GUIDANCE SYSTEM

(71) Applicant: Boston Scientific Scimed, Inc., Maple Grove, MN (US)

(72) Inventors: Christopher J. Nycz, Holden, MA (US); Andrew Daudelin, Worcester, MA (US); Bryan Allen Clark, Forest Lake, MN (US); Aiden Flanagan, Galway (IE); Longquan Chen, Lexington, MA (US); Kian S. Lim, Shrewsbury, MA (US); Niraj Prasad Rauniyar, Plymouth, MN (US)

(73) Assignee: Boston Scientific Scimed, Inc., Maple Grove, MN (US)

(21) Appl. No.: 18/955,308

(22) Filed: Nov. 21, 2024

Related U.S. Application Data
(60) Provisional application No. 63/602,147, filed on Nov. 22, 2023.

(51) Int. Cl. A61B 17/34; A61B 90/00

(52) U.S. Cl. CPC A61B 17/3403; A61B 90/37; A61B 2090/376

(57) ABSTRACT

Medical devices and methods for using medical devices are disclosed. An example method for performing percutaneous nephrolithotomy includes registering an optical tracking system to a global coordinate system, registering a fluoroscope to the global coordinate system, generating a first image of a treatment area of a patient using the fluoroscope, generating a second image of the treatment area of the patient using the fluoroscope, selecting a target treatment point within the treatment area on the first image, the second image or both the first image and the second image and overlaying a medical instrument proposed guidance line on the first image, the second image or both the first image and the second image, wherein the medical instrument proposed guidance line depicts a proposed insertion pathway from an insertion point on the patient's body to the target treatment point.

### CROSS-REFERENCE TO RELATED APPLICATIONS

This application claims the benefit of priority under 35 U.S.C. § 119 of U.S. Provisional Application No. 63/602,147, filed Nov. 22, 2023, the entire disclosure of which is hereby incorporated by reference.

### TECHNICAL FIELD

The disclosure is directed to surgical needle guidance systems. More particularly, the disclosure is directed to surgical needle guidance systems for use in a percutaneous nephrolithotomy (PCNL) medical procedure.

### BACKGROUND

Percutaneous Nephrolithonomy (PCNL) is a minimally invasive surgery for removing kidney stones through an access sheath inserted through a patient's back and into the kidney. Using a fluoroscope for guidance, a physician initially gains access to the target site by first inserting a needle into the patient and then dilating a needle track for placement of the access sheath. Once the access sheath is positioned appropriately, a medical device (e.g., nephroscope, forceps, etc.) may be passed through the access sheath and advanced to the target kidney stones. The medical device may be utilized to remove kidney stones. Depending on the complexity of the procedure, the patient may be subject to prolonged radiation exposure as the clinician uses the fluoroscope to provide real-time visualization of the target kidney stones. Of the known surgical guidance systems and methods, there is an ongoing need to provide alternative configurations of surgical guidance systems which limit the radiation exposure to a patient while maintaining effective visualization of the target treatment site, as well as methods of operating such surgical guidance systems. Surgical guidance systems which limit the radiation exposure to a patient while maintaining effective visualization of the target treatment site are disclosed herein.

### SUMMARY

This disclosure provides design, material, manufacturing method, and use alternatives for medical devices. An example method for performing percutaneous nephrolithotomy includes registering an optical tracking system to a global coordinate system, registering a fluoroscope to the global coordinate system, generating a first image of a treatment area of a patient using the fluoroscope, generating a second image of the treatment area of the patient using the fluoroscope, selecting a target treatment point within the treatment area on the first image, the second image or both the first image and the second image and overlaying a medical instrument proposed guidance line on the first image, the second image or both the first image and the second image, wherein the medical instrument proposed guidance line depicts a proposed insertion pathway from an insertion point on the patient's body to the target treatment point.

Alternatively or additionally to any of the examples above, wherein the first image, the second image or both the first and the second image are two-dimensional.

Alternatively or additionally to any of the examples above, wherein the first image, the second image or both the first and the second image are three-dimensional.

Alternatively or additionally to any of the examples above, wherein the medical instrument includes an access needle.

Alternatively or additionally to any of the examples above, further comprising registering the access needle to the global coordinate system.

Alternatively or additionally to any of the examples above, further comprising overlaying a medical instrument alignment line on the first image, the second image or both the first image and the second image, wherein the medical instrument alignment line depicts an alignment of the medical instrument relative to the treatment area.

Alternatively or additionally to any of the examples above, further comprising manipulating the medical instrument to align the medical instrument proposed guidance line with the medical instrument alignment line.

Alternatively or additionally to any of the examples above, wherein the first image is generated by the fluoroscope when the fluoroscope is positioned at a first angular orientation, and wherein the second image is generated by the fluoroscope when the fluoroscope is positioned at a second angular orientation different from the first angular orientation.

Alternatively or additionally to any of the examples above, wherein the second angular orientation is offset from the first angular orientation by 25-35 degrees.

Alternatively or additionally to any of the examples above, wherein registering an optical tracking system to a global coordinate system and registering a fluoroscope to the global coordinate system further includes simultaneously using the fluoroscope to generate the first image and the second image while the tracking system acquires the positions of a fiducial marker included in the first image and the second image.

Alternatively or additionally to any of the examples above, further comprising utilizing a transformation matrix to register a coordinate system of the fluoroscope with a coordinate system of the tracking system within the global coordinate system.

Alternatively or additionally to any of the examples above, wherein first image, the second image or both the first image and the second image further includes a guidance line confidence indicator, and wherein the guidance line confidence indicator conveys a relative confidence level in the accuracy of the medical instrument proposed guidance line.

Alternatively or additionally to any of the examples above, wherein a physician manually selects the target treatment point on the first image, the second image or both the first image and the second image.

Alternatively or additionally to any of the examples above, wherein a computer selects the target treatment point on the first image, the second image or both the first image and the second image.

Another example method for performing percutaneous nephrolithotomy includes calibrating an access needle using a calibration tool, wherein the calibration tool is registered to a global coordinate system. The method also includes registering an optical tracking system to a global coordinate system, registering a fluoroscope to the global coordinate system, generating a first image of a treatment area of a patient using the fluoroscope, generating a second image of the treatment area of the patient using the fluoroscope, simultaneously generating a first image and a second image of a treatment area of a patient using the fluoroscope while the tracking system acquires the positions of a fiducial marker within a field of view of the fluoroscope, selecting a target treatment point with the treatment area on the first image, the second image or both the first image and the second image and overlaying a medical instrument proposed guidance line on the first image, the second image or both the first image and the second image, wherein the medical instrument proposed guidance line depicts a proposed insertion pathway from an insertion point on the patient's body to the target treatment point.

Alternatively or additionally to any of the examples above, wherein the first image, the second image or both the first and the second image are two-dimensional.

Alternatively or additionally to any of the examples above, wherein the first image, the second image or both the first and the second image are three-dimensional.

Alternatively or additionally to any of the examples above, further comprising overlaying a medical instrument alignment line on the first image, the second image or both the first image and the second image, wherein the medical instrument alignment line depicts an alignment of the medical instrument relative to the treatment area.

Alternatively or additionally to any of the examples above, further comprising manipulating the medical instrument to align the medical instrument proposed guidance line with the medical instrument alignment line.

An example needle guidance system for performing percutaneous nephrolithotomy includes a processor coupled to a fluoroscope, an optical tracking system and a display. Further, the optical tracking system is registered to a global coordinate system, the fluoroscope is registered to the global coordinate system, the fluoroscope is configured to generate a first image and a second image of a treatment area of a patient and the display is configured to permit a physician to select a target treatment point with the treatment area on the first image, the second image or both the first image and the second images. Further, after selecting the target treatment point, the processor is configured to overlay a medical instrument proposed guidance line on the first image, the second image or both the first image and the second images, wherein the medical instrument proposed guidance line depicts a proposed insertion pathway from an insertion point on the patient's body to the target treatment point.

The above summary of some example embodiments is not intended to describe each disclosed embodiment or every implementation of the disclosure.

### BRIEF DESCRIPTION OF THE DRAWINGS

The disclosure may be more completely understood in consideration of the following detailed description of various embodiments in connection with the accompanying drawings, in which:

FIG. 1 is a perspective view of multiple components of an example needle guidance system;

FIG. 2 illustrates multiple components of an example needle guidance system positioned within a global reference frame;

FIG. 3 illustrates an example insertion needle positioned adjacent to an example needle calibration tool;

FIG. 4 illustrates an example fluoroscopic system in a first angular orientation;

FIG. 5 illustrates an example image taken by the fluoroscopic system of FIG. 4;

FIG. 6 illustrates the fluoroscopic system of FIG. 4 being rotated through an angle;

FIG. 7 illustrates the fluoroscopic system of FIG. 4 in a second angular orientation;

FIG. 8 illustrates an example image taken by the fluoroscopic system of FIG. 7;

FIG. 9 illustrates example guidance displays of the fluoroscopic system of FIG. 1;

FIG. 10 illustrates a depth camera attached to the fluoroscopic system of FIG. 1.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

All numeric values are herein assumed to be modified by the term “about”, whether or not explicitly indicated. The term “about” generally refers to a range of numbers that one of skill in the art would consider equivalent to the recited value (e.g., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

Percutaneous Nephrolithonomy (PCNL) is a minimally invasive surgery for removing kidney stones through an access sheath inserted through a patient's back and into the kidney. Using a fluoroscope for guidance, a physician initially gains access to the target site by first inserting a needle into the patient and then dilating a needle track for placement of the access sheath. Traditionally, placement of the needle is accomplished with the “bullseye” technique, which involves first lining up the axis of the needle in a fluoroscope image so that it intersects the desired calyx of the kidney, after which the fluoroscope is rotated approximately 20-30 degrees while the needle is inserted to the desired depth. The first viewpoint from the fluoroscope image identifies the needle path, and the second viewpoint allows for monitoring the depth of the needle during the insertion.

PCNL access is typically accomplished by an interventional radiologist prior to a urologist performing the stone extraction. Access by a radiologist, however, has the potential to add time and complexity to the procedure and may result in greater complications. Therefore, in some instances it may be beneficial to provide alternative configurations of surgical guidance systems which simplify PCNL access with improved navigation aids that could help lower the learning curve of the procedure and limit radiation exposure to patients undergoing PCNL. For example, designing a PCNL needle guidance system to present continuous overlay of a current needle position relative to static fluoroscope images enables the physician to continually visualize the needle tract with minimal fluoroscope exposure. Further, designing a PCNL needle guidance system to include a camera mounted to a fluoroscope to provide 3D localization and visualization may simplify PCNL access and limit radiation exposure to patients undergoing PCNL. Of the known surgical guidance systems and methods, there is an ongoing need to provide alternative configurations of surgical guidance system, as well as methods of operating such surgical guidance systems.

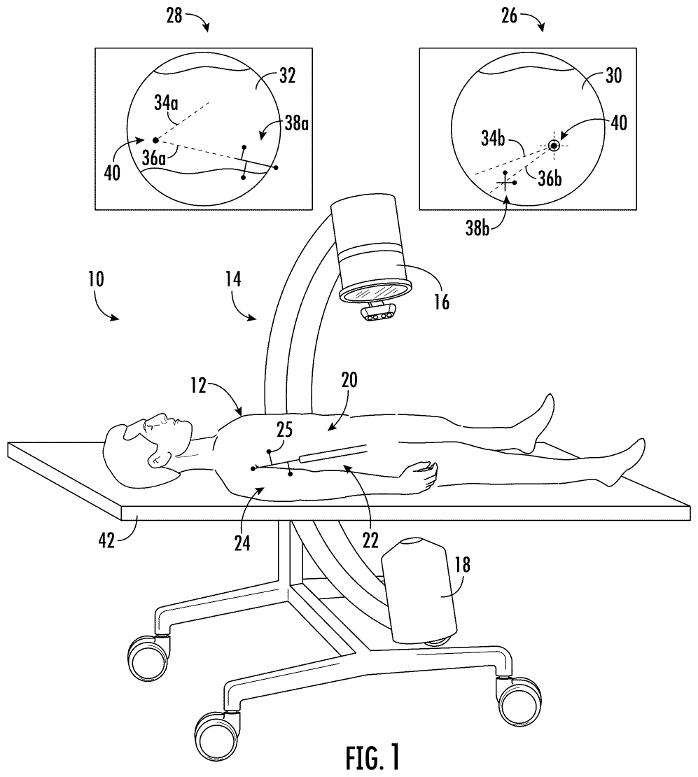

FIG. 1 is a perspective view of an illustrative guided needle insertion system 10. FIG. 1 illustrates a patient 12 positioned within an example operating theatre whereby the patient is undergoing an example medical procedure. For example, FIG. 1 illustrates that the patient 12 may be undergoing a percutaneous nephrolithotomy, whereby the patient 12 is positioned on operating table 42 which is coupled to a fluoroscope 14 (e.g., a C-arm machine). Fluoroscopy is a medical imaging procedure which displays real-time, continuous X-ray imaging on one or more displays. During a fluoroscopy procedure, an X-ray beam is passed from a radiation source 18 (coupled to the fluoroscope 14), through the patient's 12 body whereby the portions of the radiation not “blocked” by various structures of the patient's anatomy are received by the radiation detector 16 (coupled to the fluoroscope 14). The resulting X-ray image is then transmitted to one or more displays 26, 28 so the movement of a body part, a medical instrument and/or contrast agent through the patient's body can be seen in detail. Fluoroscopy carries some risks, as do other X-ray procedures. The radiation dose the patient receives varies depending on the individual procedure. In some instances, fluoroscopy can result in the patient being exposed to relatively high radiation doses, especially for complex interventional procedures (such as PCLN) which may require fluoroscopy be administered for a long period of time when the physician is determining a target kidney stone and/or inserting and tracking the needle to the targeted kidney stones.

As discussed herein, designing a PCNL needle guidance system that simplifies the PCNL needle insertion and tracking procedure may limit the total radiation exposure to patients undergoing PCNL. Accordingly, FIG. 1 further illustrates a first display 26 (e.g., monitor, screen, visual display unit, etc.) which shows a first two-dimensional image 30 of a target treatment site (e.g., kidney, kidney stone) of the patient 12. Further, FIG. 1 also illustrates a second display 28 (e.g., monitor, screen, visual display unit, etc.) which shows a second two-dimensional image 32 of the target treatment site (e.g., kidney, kidney stone) of the patient 12. It can be appreciated that each of the displays 26, 28 may be mounted in the operating theater such that a physician performing the PCNL on the patient 12 can access the patient's anatomy while also maintaining visibility to each of the first display 26 and the second display 28. It can be further appreciated from FIG. 1 that the first two-dimensional image 30 and the second two-dimensional image 32 may represent the same anatomical target site, yet differ because each of the images 26, 28 was taken at a different angle by the fluoroscope 14. For example, the first two-dimensional image 30 may have been taken with the fluoroscope 14 at a first position (e.g., 0 degree relative angle) while the second two-dimensional image 32 may have been taken with the fluoroscope 14 at a second position (e.g., 30 degree relative angle) relative to the first position. In some examples, the first image 26 may correspond to an on end “Bullseye” view while the second image 28 may and represent an oblique view.

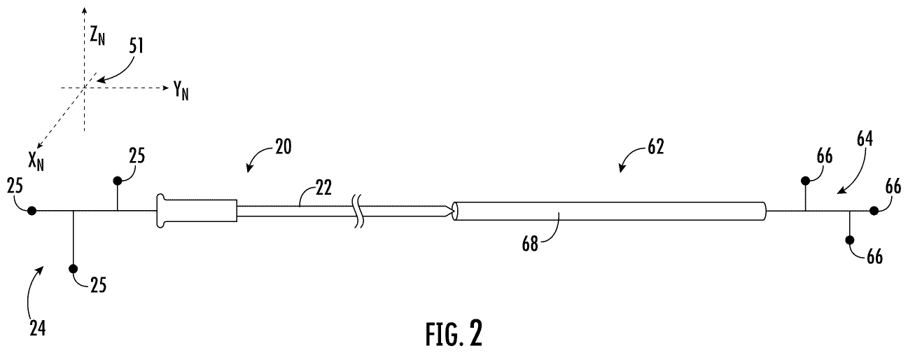

As will be discussed in greater detail below, FIG. 1 further illustrates that each of the static first two-dimensional image 30 and the static second two-dimensional image 32 may include a live-view overlay of the needle 20 being utilized in the PCNL procedure. It can be appreciated that to create the overlay of the needle 20 on the static first two-dimensional image 30 and the static second two-dimensional image 32, one or more fiducial markers 25 may be coupled to the needle 20. Attaching the fiducial markers 25 to the needle 20 may permit both an optical tracking system 52 (shown in FIG. 2) to work in cooperatively with the fluoroscope 14 to overlay the needle 20 on the static first two-dimensional image 30 and the static second two-dimensional image 32. Additionally, FIG. 1 illustrates that, in some examples, each display 26, 28 of the needle guidance system described herein may include an overlay of a “proposed” needle pathways 34a, 34b on each of the static first two-dimensional image 30 and the static second two-dimensional image 32, respectively, whereby the proposed needle pathways 34a, 34b represent a calculated, optimized needle pathway that a physician should follow when advancing the needle 20 post-insertion to have the needle tip reach the target site 40 (e.g., kidney stone, etc.). Additionally, in some examples, each display 26, 28 of the needle guidance system described herein may also include a live-view overlay of a “current” needle alignment line 36a, 36b on each of the static first two-dimensional image 30 and the static second two-dimensional image 32, respectively, whereby the current needle alignment line 36a, 36b represents the instant alignment of the needle 20 during the live procedure. It can be further appreciated that the physician may manipulate the position of the needle 20 such that the current needle alignment line 36a, 36b overlays the proposed needle pathway 34a, 34b to optimally position the needle for insertion and advancement to the target site 40.

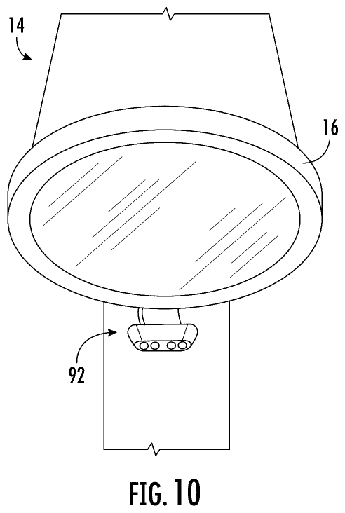

As discussed herein, reductions in radiation exposure for the patient and physician are possible by removing the need for continuous fluoroscopy during access. The ability to present continuous overlay of the current needle position relative to static fluoroscope images enables the physician to continually visualize the needle tract with minimal fluoroscope exposure. The fluoroscope usage to gain access could be on the order of seconds as opposed to minutes, as is the current standard. It can be appreciated that in order for the needle guidance system to generate the proposed needle pathways 34a, 34b (show in FIG. 1), a series of calibration steps may need to be performed to co-register the needle to the optical tracking system and the radiation source of the fluoroscope. The following calibration steps described relative to FIGS. 2-8 may be performed pre-operatively. As will be described with respect to FIG. 10, after the calibration steps are completed, fluoroscopic images of the patient 12 may be taken which may be preserved as static images upon which the proposed needle pathways and current needle alignment lines described herein may be superimposed.

FIG. 2 illustrates that a first calibration step may include calibrating the insertion needle using a calibration tool 62. For example, it can be appreciated that in some instances, the shaft 22 of the insertion needle 20 may not be perfectly straight. Accordingly, a calibration step may need to be performed which calibrates the tip of the needle 20 to one or more fiducial markers 25 attached to a proximal end region of the needle 20 (FIG. 2 illustrates the fiducial markers 25 attached to an end of the needle 20 opposite the needle tip). It can be appreciated that the fiducial markers 25 may be registered to a needle coordinate system 51 whereby the position of each of the fiducial markers 25 is aligned with the axes X_{N}, Y_{N}, Z_{N} of the need coordinate system 51.

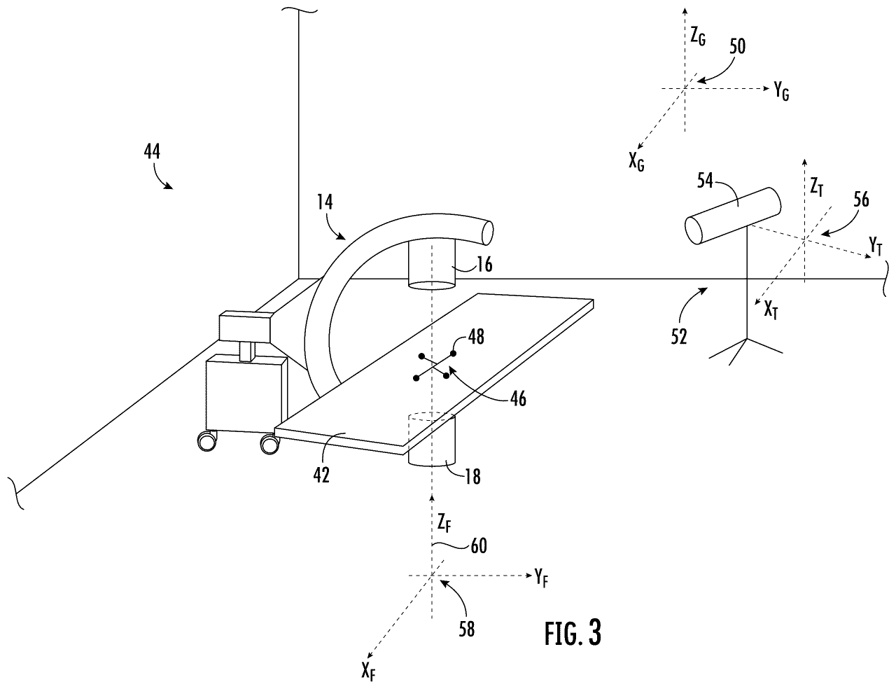

FIG. 2 further illustrates that the calibration tool 62 may include one or more fiducial markers 66 attached to a proximal end region of the calibration tool 62. Further, it can be appreciated that the fiducial markers 66 of the calibration tool 62 may be registered within a global coordinate system 50 (shown in FIG. 3). It can be appreciated that the fiducial markers 66 may be registered to the global coordinate system 50 whereby the position of each of the fiducial markers 66 is aligned with the axes X_{G}, Y_{G}, Z_{G} of the need coordinate system 50.

Additionally, FIG. 2 illustrates that calibration of the insertion needle 20 may including placing the tip of the needle in contact with the distal end of the calibration tool 62. It can be appreciated that the calibration tool shaft 68 may be manufactured with precise tolerances such that the distal end of the calibration tool 62 may be precisely aligned within the global coordinate system 50. Accordingly, by placing the tip of the needle 20 in contact with the distal end of the calibration tool 62 a transformation and/or curve fitting may be utilized which may register both the fiducial markers 25 and the tip of the insertion needle 20 within the global coordinate system 50. It can be appreciated that after this calibration step is performed, an optical imaging system which is registered with the global coordinate system 50 may be able to determine the location of the tip of the needle 20 via tracking the fiducial markers 25 attached to the needle 20.

It can be further appreciated that the insertion needle calibration step described with respect to FIG. 2 may also be carried out using alternative methods. For example, the insertion needle 20 calibration may be performed via pivot calibration, whereby the tip of the needle 20 is held stationary within a fixture (e.g., a block) having a known, fixed position within the coordinate system. Further, while holding the tip of the needle 20 stationary, the fiducial markers 25 attached to the needle 20 are moved around relative to the fixture and their position is captured via cameras which are also registered to the fixed coordinate system for which the position of the fixture is registered. Accordingly, the location of the tip of the needle 20 may be determined within the reference frame of the fixture using the pivot calibration method.

In some examples, any of the fiducial markers disclosed herein may include reflective passive markers whereby IR light from a motion capture system may calculate the position of the passive markers. In other examples, any of the fiducial markers disclosed herein may include one or more AprilTags® (e.g., 2D barcode or simplified QR code) that, when photographed or imaged, can be utilized to determine the location and orientation of the AprilTag® and insertion need to which it is attached. In yet other examples, any of the fiducial markers disclosed herein may include magnetic trackers that can be utilized to determine the location and orientation of the insertion need to which they are attached.

FIG. 3 illustrates that a second calibration step may include calibrating the radiation source 18 of the fluoroscope with the optical tracking system 52. FIG. 3 illustrates that the optical tracking system 52 may be positioned away from the fluoroscope 14 and held in a fixed (e.g., stationary) position relative to both the fluoroscope and the operating theater. The position of the tracking system 52 may be aligned with the axes X_{T}, Y_{T}, Z_{T} of a tracking coordinate system 56. Accordingly, it can be appreciated that a transformation may be utilized to register the tracking coordinate system 56 with the global coordinate system 50 discussed herein.

Additionally, FIG. 3 illustrates the radiation source 18 attached to the fluoroscope 14, whereby the position of the radiation source 18 may be known relative the axes X_{F}, Y_{F}, Z_{F} of a fluoroscope coordinate system 58. Additionally, it can be appreciated the additional fluoroscope parameters may be known, measured and/or acquired, which may be necessary for co-registration of the fluoroscope coordinate system 58 and the tracking system 52. These fluoroscope parameters may include the focus length of the fluoroscope and/or pixel scaling parameters (e.g., pixel-to-world scaling parameters). FIG. 3 further illustrates that calibrating the radiation source 18 to the tracking system 52 may include registering an x-ray image taken of multiple markers (e.g., four or more) fiducial markers 48 placed within the field view of both the radiation source 18 and tracking system 52. The fiducial markers 48 may be known within the global coordinate system 50. Further, the optical tracking system may visualize the markers 48 and determine their position within the global coordinate system 50.

To localize the target position in three-dimensional space, two images may be acquired with the fluoroscope 14. As described below, the two images may be acquired from two distinct viewpoints by moving the fluoroscope 14 20-40 degrees (e.g., 30 degrees) in its orbital motion. The angle between the two viewpoints may be measured using various techniques. For example, an orientation sensor (potentially incorporated into a component of the fluoroscope) which includes an accelerometer, gyroscope, and magnetometer may be used to measure the angular change in position of the fluoroscope 14. Additionally, measurement with a potentiometer or encoder mechanically coupled to the orbital motion of the fluoroscope 14 may be used to measure the angular change in position of the fluoroscope 14. Further, an external marker based visual tracking system may be used to measure the angular change in position of the fluoroscope 14.

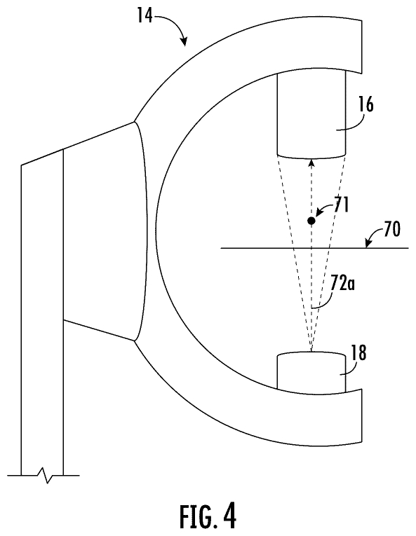

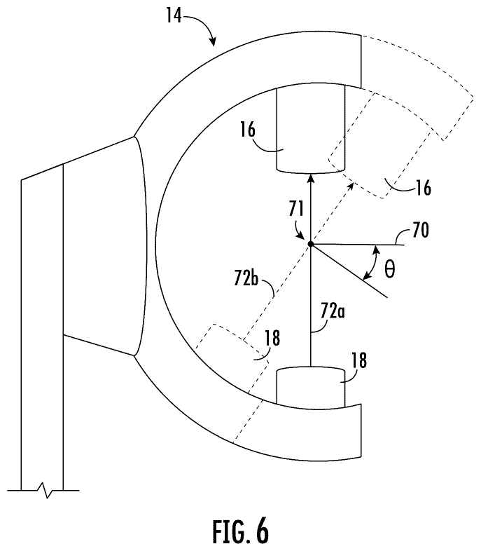

FIG. 4 illustrates a side view of the fluoroscope 14 including the radiation source 18 and the detector 16. It can be appreciated from FIG. 4 that x-ray emitted from the radiation source 18 may extend from the radiation source 18 in a cone-shaped pattern toward the detector. Further, FIG. 4 illustrates an example vector of a single x-ray beam 72a emitted from the radiation source 18. Further, FIG. 4 illustrates an example target point 71 with is aligned along the x-ray beam 72a. It may be appreciated that the target 71 shown in FIG. 4 may represent a fiducial marker 48 (shown in FIG. 2) discussed herein. It can be further appreciated that the x-ray beam 72a may be aligned substantially perpendicular to the horizontal axis 70 shown in FIG. 4. For simplicity purposes relative to FIG. 4 and FIG. 6, it will be assumed herein that the x-ray beam 72a is aligned at 0 degrees relative to the horizontal axis 70 shown in FIG. 4 and FIG. 6.

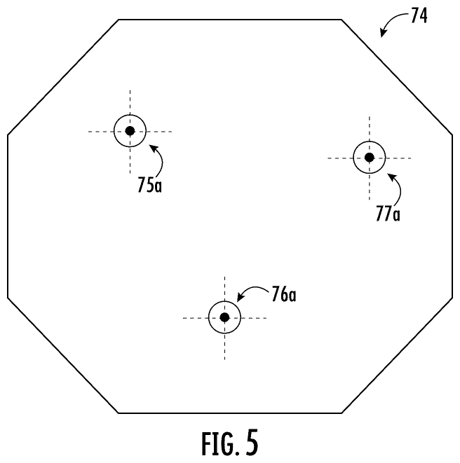

FIG. 5 illustrates an example two-dimensional image 74 depicting the coordinates 75a, 76a, 77a of three fiducial markers 48 shown in FIG. 2. As discussed herein, the relative position of these markers 48 would be established by x-ray beams emitted from the radiation source 18 when the fluoroscope is positioned at 0 degrees relative to the horizontal axis 70.

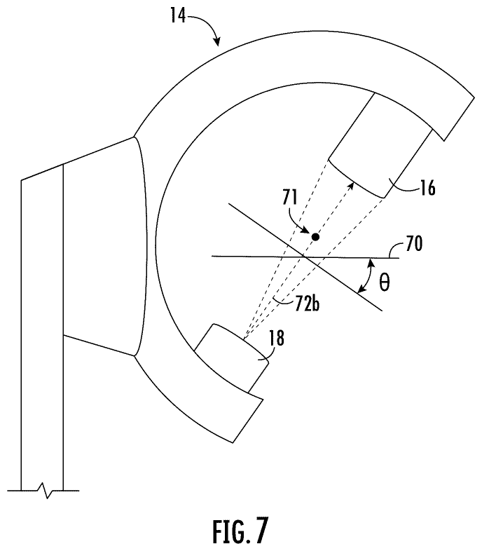

FIG. 6 illustrates a side view of the fluoroscope 14 including the radiation source 18 and the detector 16. However, FIG. 6 illustrates that the fluoroscope 14 being rotated through an angle θ relative to the horizontal axis 70. It can be appreciated that a position sensor may be attached to the fluoroscope 14 to determine its position relative to the horizontal axis 70. In some examples, the fluoroscope may be rotated approximately 30 degrees relative to the horizontal axis 70. Further, FIG. 6 illustrates an example vector of a single x-ray beam 72b emitted from the radiation source 18. Further, FIG. 4 illustrates the example target point 71 aligned along the x-ray beam 72b. It may be appreciated that the target 71 (which represent the same fiducial marker 48 described with respect to FIG. 4) discussed herein. FIG. 7 illustrates the position of the fluoroscope 14 after having been rotated approximately 30 degrees relative to the horizontal axis 70.

Further, by rotating the fluoroscope approximately 30 degrees between the two images described above, two distinct viewpoints are given which provide both position and depth information. The 30 degree rotation provides a large motion of the targets in the fluoroscope field of view, allowing for targeting that is less susceptible to image noise or small errors in placing the target location. After the two viewpoints are acquired, the target 71 can be located in three-dimensional space by locating the point of nearest intersection between the target vector 72a and the target vector 72b in the two images.

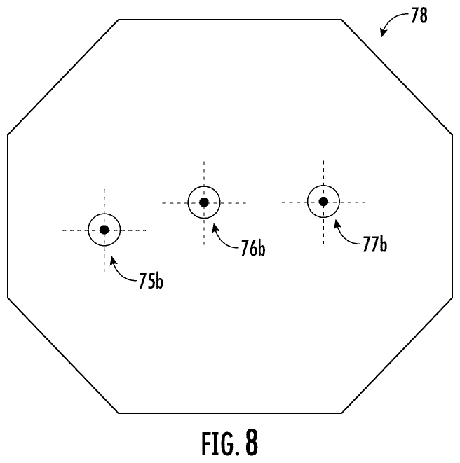

FIG. 8 illustrates another example two-dimensional image 78 depicting the coordinates 75b, 76b, 77b of the three fiducial markers 48 shown in FIG. 2. As discussed herein, the relative position of these markers 48 would be established by x-ray beams emitted from the radiation source 18 when the fluoroscope is positioned at 30 degrees relative to the horizontal axis 70. Accordingly, it can be appreciated that because the fluoroscope has been rotated through an angle of 30 degrees relative to its position in FIG. 4, the coordinates 75b, 76b, 77b of the three fiducial markers 48 shown in FIG. 8 may be different than the coordinates 75a, 76a, 77a of the three fiducial markers 48 shown in FIG. 5. Further, while the fluoroscope is capturing the images of the markers 48 at two different angles, the position of the markers 48 are being acquired by the tracking system 52. Accordingly, by viewing the fiducial markers 48 in two different fluoroscope images taken when the fluoroscope 14 is positioned at two different angles (e.g., 0 degrees and 30 degrees relative to the horizontal axis 70) simultaneously with the tracking system acquiring the positions of the markers 48 and the known (or measured) fluoroscope parameters (including the fluoroscope focus length and the scaling parameters), an algorithm may be developed to calculate a transformation matrix which may co-register the fluoroscope coordinate system 18 (and images derived from the radiation source 18 of the fluoroscope 14) with the tracking coordinate system 56 within the global coordinate system 50 discussed herein. It can be further appreciated that after the fluoroscope coordinate system 18 (and images derived from the radiation source 18 of the fluoroscope 14) is co-registered with the tracking coordinate system 56 within the global coordinate system 50, the fiducial markers 25 attached to the needle 20 may be visualized and tracked by the tracking system 52 in addition to being visualized on images derived from the fluoroscope 14.

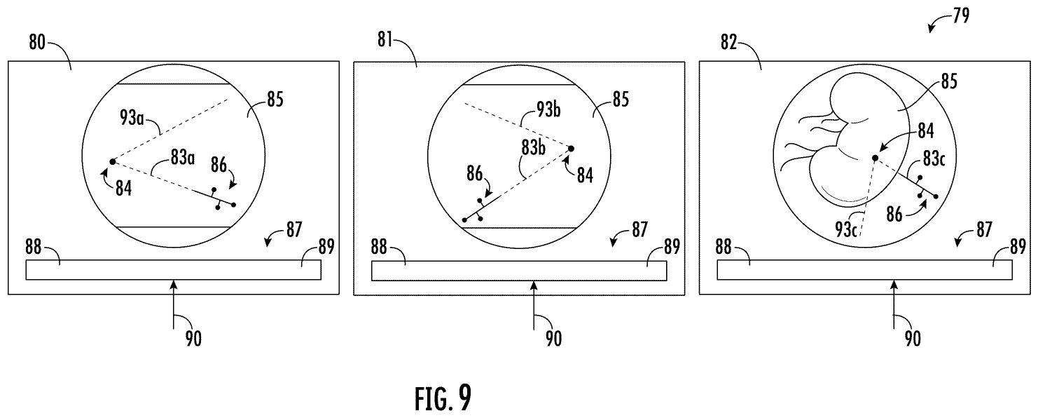

FIG. 9 illustrates example group 79 of displays 80, 81 showing an example two-dimensional image and an example display 82 showing an example three-dimensional image of a patient's target site (e.g., kidney 85) during a medical procedure (e.g., a PCNL procedure). The displays 80, 81, 82 shown in FIG. 9 may represent variations of the displays 26, 28 shown and described herein relative to FIG. 2. For example, each of the displays 80, 81, 82 may be mounted in the operating theater such that a physician performing the PCNL on the patient 12 can access the patient's anatomy while also maintaining visibility to each of the displays 80, 81, 82.

In some examples, after the images appear on the displays 80, 81, 82, a physician may be able to actively select a target site position on the display. The target site may represent the anatomical location within the patient 12 for which the physician would like the tip of the insertion needle 20 to contact. For example, to access and remove a kidney stone within the patient 12, the physician 12 would visualize the stone one or more of the displays 80, 81, 82 and select the kidney stone as the target treatment site. In other examples, the needle guidance system disclosed herein may include an image recognition algorithm which automatically selects or provides suggestions for potential target treatment sites based on pre-programmed parameters. The image recognition software may utilize labeled and segmented images from prior PCNL procedures to provides suggestions for potential target treatment sites.

After the physician selects the target treatment site on the displays, the guidance system may calculate and overlay a proposed needle pathway on each of the static first two-dimensional displays 80, 81 and the static three-dimensional image on the display 82, respectively, whereby the proposed needle pathways represent a calculated, optimized needle pathway that a physician should follow when advancing the needle 20 to have the needle tip reach the target site (e.g., kidney stone, etc.). The displays 80, 81, 82 may also include a live-view overlay of a current needle alignment line on each of the static first two-dimensional displays 80, 81 and the static three-dimensional image on the display 82, respectively, whereby the current needle alignment line represents the instant alignment of the needle 20 during the live procedure. For example, the two-dimensional display 80 illustrates a proposed needle pathway 93a and the fiducial markers 86 of an insertion needle positioned along a current needle alignment line 83a, whereby each of the proposed needle pathway 93a and the current needle alignment line 83a are superimposed over an image (taken by the fluoroscope 14 at a first angle) of a patient 12 and point to a target site 84 within the patient 12. Further, the two-dimensional display 81 illustrates a proposed needle pathway 93b and the fiducial markers 86 of the insertion needle positioned along a current needle alignment line 83b, whereby each of the proposed needle pathway 93b and the current needle alignment line 83b are superimposed over an image (taken by the fluoroscope 14 at a second angle different than the first angle, as described herein) of a patient 12 and point to a target site 84 within the patient 12. Further yet, the three-dimensional display 82 illustrates a proposed needle pathway 93c and the fiducial markers 86 of the insertion needle positioned along a current needle alignment line 83c, whereby each of the proposed needle pathway 93c and the current needle alignment line 83c are superimposed over a three-dimensional image of a target site (e.g., kidney) of patient 12 and point to a target site 84 within the patient 12. As discussed herein, it can be appreciated that the physician may manipulate the position of the needle in the displays 80, 81, 82 such that the current needle alignment lines 83a, 83b, 83c overlay the proposed needle pathways 93a, 93b, 93c, respectively to optimally position the needle for insertion and advancement to the target site 84.

FIG. 9 further illustrates that, in some examples, one or more of the displays 80, 81, 82 may include a “confidence indicator” 87 which provides the physician with a visual indication of the confidence that the needle guidance system has in the proposed needle alignment pathway 93a, 93b, 93c, respectively. FIG. 9 illustrates that the indicator 87 may include a spectrum having a first end 88 and a second end 89. The first end 88 may represent a low confidence that a given proposed needle alignment pathway 93a, 93b, 93c is accurately depicted. Additionally, the second end 89 of the spectrum may be represent a high confidence that a given proposed needle alignment pathway 93a, 93b, 93c is accurately depicted. Further, in some examples, the indicator 87 may include an arrow which provides the physician with a confidence indication at a point somewhere along the spectrum between the first end 88 (low confidence) and the second end (high confidence) that a given proposed needle alignment pathway 93a, 93b, 93c is accurately depicted.

FIG. 10 illustrates that, in some examples, a three-dimensional depth camera 92 may be fixedly attached to one or more structures (e.g., the detector) of the fluoroscope 14. In some instances, the camera may be calibrated to the fluoroscope 14 field of view and provide continuous three-dimensional positional tracking of the PCNL access needle. Similar to that discussed herein with respect to FIG. 4 and FIG. 6-7, two still images may be acquired using the fluoroscope 14 at two different viewpoints (approximately 30 degrees apart). The target site (e.g., stone or calyx) where the insertion needle is desired to be placed may be selected by the physician by clicking on the target in each fluoroscope image. Through these two data points, the three-dimensional position of the target can be calculated. The navigation software may then provide continuous three-dimensional visualization of the target position relative to the current needle position from multiple viewpoints, simplifying the navigation process.

In addition to tracking the needle that the surgeon manipulates, the three-dimensional depth camera 92 may also track the patient on the table. Additionally, during the 30 degree orbital rotation of the fluoroscope 14 needed for targeting the three-dimensional camera 92 may generate a more complete three-dimensional model of the patient. This three-dimensional model can be used for visualization of the entry point on the patient's body through the overlay of a target on the current RGB video feed of the surgical field provided by the three-dimensional camera. Further, the three-dimensional model can be used to measure patient motion in order to alert the surgeon that the targeting images are no longer valid. Further, the three-dimensional model can be used for measuring patient motion in order to update the targeting information for the new patient position. Further, the three-dimensional model can be used for measurement of skin deformation for approximating deformation and motion of the target internal to the patient.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A method for performing percutaneous nephrolithotomy, the method comprising:
registering an optical tracking system to a global coordinate system;
registering a fluoroscope to the global coordinate system;
generating a first image of a treatment area of a patient using the fluoroscope;
generating a second image of the treatment area of the patient using the fluoroscope;
selecting a target treatment point within the treatment area on the first image, the second image or both the first image and the second image;
overlaying a medical instrument proposed guidance line on the first image, the second image or both the first image and the second image, wherein the medical instrument proposed guidance line depicts a proposed insertion pathway from an insertion point on the patient's body to the target treatment point.

2. The method of claim 1, wherein the first image, the second image or both the first and the second image are two-dimensional.

3. The method of claim 1, wherein the first image, the second image or both the first and the second image are three-dimensional.

4. The method of claim 1, wherein the medical instrument includes an access needle.

5. The method of claim 4, further comprising registering the access needle to the global coordinate system.

6. The method of claim 1, further comprising overlaying a medical instrument alignment line on the first image, the second image or both the first image and the second image, wherein the medical instrument alignment line depicts an alignment of the medical instrument relative to the treatment area.

7. The method of claim 6, further comprising manipulating the medical instrument to align the medical instrument proposed guidance line with the medical instrument alignment line.

8. The method of claim 1, wherein the first image is generated by the fluoroscope when the fluoroscope is positioned at a first angular orientation, and wherein the second image is generated by the fluoroscope when the fluoroscope is positioned at a second angular orientation different from the first angular orientation.

9. The method of claim 8, wherein the second angular orientation is offset from the first angular orientation by 25-35 degrees.

10. The method of claim 1, wherein registering an optical tracking system to a global coordinate system and registering a fluoroscope to the global coordinate system further includes simultaneously using the fluoroscope to generate the first image and the second image while the tracking system acquires the positions of a fiducial marker included in the first image and the second image.

11. The method of claim 10, further comprising utilizing a transformation matrix to register a coordinate system of the fluoroscope with a coordinate system of the tracking system within the global coordinate system.

12. The method of claim 11, wherein first image, the second image or both the first image and the second image further includes a guidance line confidence indicator, and wherein the guidance line confidence indicator conveys a relative confidence level in the accuracy of the medical instrument proposed guidance line.

13. The method of claim 1, wherein a physician manually selects the target treatment point on the first image, the second image or both the first image and the second image.

14. The method of claim 1, wherein a computer selects the target treatment point on the first image, the second image or both the first image and the second image.

15. A method for performing percutaneous nephrolithotomy, the method comprising:
calibrating an access needle using a calibration tool, wherein the calibration tool is registered to a global coordinate system;
registering an optical tracking system to a global coordinate system;
registering a fluoroscope to the global coordinate system;
generating a first image of a treatment area of a patient using the fluoroscope;
generating a second image of the treatment area of the patient using the fluoroscope;
simultaneously generating a first image and a second image of a treatment area of a patient using the fluoroscope while the tracking system acquires the positions of a fiducial marker within a field of view of the fluoroscope;
selecting a target treatment point with the treatment area on the first image, the second image or both the first image and the second image;
overlaying a medical instrument proposed guidance line on the first image, the second image or both the first image and the second image, wherein the medical instrument proposed guidance line depicts a proposed insertion pathway from an insertion point on the patient's body to the target treatment point.

16. The method of claim 15, wherein the first image, the second image or both the first and the second image are two-dimensional.

17. The method of claim 15, wherein the first image, the second image or both the first and the second image are three-dimensional.

18. The method of claim 15, further comprising overlaying a medical instrument alignment line on the first image, the second image or both the first image and the second image, wherein the medical instrument alignment line depicts an alignment of the medical instrument relative to the treatment area.

19. The method of claim 18, further comprising manipulating the medical instrument to align the medical instrument proposed guidance line with the medical instrument alignment line.

20. A needle guidance system for performing percutaneous nephrolithotomy, comprising:
a processor coupled to a fluoroscope, an optical tracking system and a display;
wherein the optical tracking system is registered to a global coordinate system;
wherein the fluoroscope is registered to the global coordinate system;
wherein the fluoroscope is configured to generate a first image and a second image of a treatment area of a patient;
wherein the display is configured to permit a physician to select a target treatment point with the treatment area on the first image, the second image or both the first image and the second images, and wherein after selecting the target treatment point, the processor is configured to overlay a medical instrument proposed guidance line on the first image, the second image or both the first image and the second images, wherein the medical instrument proposed guidance line depicts a proposed insertion pathway from an insertion point on the patient's body to the target treatment point.
